SEQUENCE LISTING (11 sequences)

SEQ ID NO: 1 - protein, 20 aa, Artificial Sequence
  synthetic catalytic domain from v raf murine sarcoma viral oncogene homologue B1, amino acids 506-525 (BRAF 506-525)
RKTRHVNILLFMGYSTKPQL

SEQ ID NO: 2 - protein, 20 aa, Artificial Sequence
  synthetic catalytic domain from v raf murine sarcoma viral oncogene homologue B1, amino acids 656-675 (BRAF 656-675)
YSNINNRDQIIFMVGRGYLS

SEQ ID NO: 3 - protein, 466 aa, Artificial Sequence
  synthetic vimentin (protein) citrullinated (recombinant)
MSTXSVSSSSYXXMFGGPGTASXPSSSXSYVTTSTXTYSLGSALXPSTSXSLYASSPGGVYATXSSAVXLXSSVPGVXLLQDSVDFSLADAINTEFKNTXTNEKVELQELNDXFANYIDKVXFLEQQNKILLAELEQLKGQGKSXLGDLYEEEMXELXXQVDQLTNDKAXVEVEXDNLAEDIMXLXEKLQEEMLQXEEAENTLQSFXQDVDNASLAXLDLEXKVESLQEEIAFLKKLHEEEIQELQAQIQEQHVQIDVDVSKPDLTAALXDVXQQYESVAAKNLQEAEEWYKSKFADLSEAANXNNDALXQAKQESTEYXXQVQSLTCEVDALKGTNESLEXQMXEMEENFAVEAANYQDTIGXLQDEIQNMKEEMAXHLXEYQDLLNVKMALDIEIATYXKLLEGEESXISLPLPNFSSLNLXETNLDSLPLVDTHSKXTLLIKTVETXDGQVINETSQHHDDLE

SEQ ID NO: 4 - protein, 21 aa, Artificial Sequence
  synthetic vimentin 415-433 cit cyclic, cyclic citrullinated vimentin variant binding RA autoantibodies
CLPNFSSLNLXETNLDSLPLC

SEQ ID NO: 5 - protein, 22 aa, Artificial Sequence
  synthetic vimentin 58-77 cit3 cyclic, cyclic citrullinated vimentin variant binding RA autoantibodies
GGCVYATXSSACVXLXSSVPGV

SEQ ID NO: 6 - protein, 21 aa, Artificial Sequence
  synthetic clusterin 231-250 cit sm1 cyclic, cyclic citrullinated clusterin variant binding RA autoantibodies
CHFSXASSCIDELFQDXFFTX

SEQ ID NO: 7 - protein, 21 aa, Artificial Sequence
  synthetic fibrinogen A 556-575 cit sm cyclic, cyclic citrullinated fibrinogen A variant binding RA autoantibodies
NTKESSSHHPGCAEFPSXGKC

SEQ ID NO: 8 - protein, 21 aa, Artificial Sequence
  synthetic fibrinogen A 616-635 cit sm cyclic, cyclic citrullinated fibrinogen A variant binding RA autoantibodies
THSTKXCHAKSXPVXGIHTSC

SEQ ID NO: 9 - protein, 21 aa, Artificial Sequence
  synthetic histone 2A H2A/a 1-20 cit sm2 cyclic, cyclic citrullinated histone 2A H2A/a variant binding RA autoantibodies
MSGXGKQGCKAXAKAKTXSSC

SEQ ID NO: 10 - protein, 20 aa, Artificial Sequence
  synthetic filaggrin 48-65 cit2v1 cyclic, cyclic citrullinated filaggrin variant binding RA autoantibodies
CTIHAHPGSXXGGXHGYHHC

SEQ ID NO: 11 - protein, 351 aa, Artificial Sequence
  synthetic catalytic domain from v raf murine sarcoma viral oncogene homologue B1, amino acids 416-766 (BRAF 416-766)
LQKSPGPQRERKSSSSSEDRNRMKTLGRRDSSDDWEIPDGQITVGQRIGSGSFGTVYKGKWHGDVAVKMLNVTAPTPQQLQAFKNEVGVLRKTRHVNILLFMGYSTKPQLAIVTQWCEGSSLYHHLHIIETKFEMIKLIDIARQTAQGMDYLHAKSIIHRDLKSNNIFLHEDLTVKIGDFGLATVKSRWSGSHQFEQLSGSILWMAPEVIRMQDKNPYSFQSDVYAFGIVLYELMTGQLPYSNINNRDQIIFMVGRGYLSPDLSKVRSNCPKAMKRLMAECLKKKRDERPLFPQILASIELLARSLPKIHRSASEPSLNRAGFQTEDFSLYACASPKTPIQAGGYGAFPVH

(19) United States
(12) Patent Application Publication
(10) Pub. No.: US 2013/0274125 A1
(43) Pub. Date: Oct. 17, 2013

(54) MULTIPLEX IMMUNOASSAY FOR RHEUMATOID ARTHRITIS AND OTHER AUTOIMMUNE DISEASES

(71) Applicant: BIO-RAD LABORATORIES INC., Hercules, CA (US)

(72) Inventors: Steven R. Binder, Berkeley, CA (US); Roger P. Walker, Benicia, CA (US); Michelle L. Delanoy, Danville, CA (US)

(73) Assignee: Bio-Rad Laboratories Inc., Hercules, CA (US)

(21) Appl. No.: 13/801,275

(22) Filed: Mar. 13, 2013

Related U.S. Application Data
(60) Provisional application No. 61/624,871, filed on Apr. 16, 2012.

(51) Int. Cl. G01N 33/68

(52) U.S. Cl. CPC G01N 33/6854
USPC 506/9; 506/18

(57) ABSTRACT

Rheumatoid arthritis and other autoimmune diseases are diagnosed by multiplex assays for antibodies to a panel of antigens that includes cyclic citrullinated peptide and at least five members of a list that includes BRAF1 506-525, BRAF2 656-675, Vimentin (protein) citrullinated, Vimentin 415-433 cit cyclic, Vimentin 58-77 cit3 cyclic, Clusterin 231-250 cit sm1 cyclic, Fibrinogen A 556-575 cit sm cyclic, Fibrinogen A 616-635 cit sm cyclic, Histones2A H2A/a 1-20 cit sm2 cyclic, Filaggrin 48-65 cit2v1 cyclic, BRAF (catalytic domain from v raf murine sarcoma viral oncogene homologue B1, amino acids 416-766).

### CROSS-REFERENCE TO RELATED PATENT APPLICATIONS

The present application claims benefit of priority to U.S. Provisional Patent Application No. 61/624,871, filed on Apr. 16, 2012, which is incorporated by reference for all purposes.

### BACKGROUND OF THE INVENTION

1. Field of the Invention

This invention resides in the field of immunodiagnostics, with particular interest in rheumatoid arthritis.

2. Description of the Prior Art

Rheumatoid arthritis is a common autoimmune disease, afflicting 0.5-1% of the world population. This systemic disease is marked by chronic inflammation of synovial joints which leads to destruction of cartilage and bone, and eventually to disability of the patient. Although not a life-threatening disease, rheumatoid arthritis can severely affect one's quality of life. Diagnosis of rheumatoid arthritis is based mostly on clinical observations, although specific serum protein and serological tests are increasingly used to assist in the diagnosis. Among the biomarkers that are frequent targets of these tests are rheumatoid factor, anti-cyclic citrullinated peptide, and C reactive protein.

Rheumatoid factor (RF) is a term used to describe a group of autoantibodies individually known as rheumatoid factors. The RF test is considered the basic screen and hallmark for the autoimmune disorder rheumatoid arthritis (RA). RF is considered an early marker since its presence is associated with an increased risk of developing RA in people with mild arthritic symptoms. Rheumatoid factor includes three subclasses that react with the crystallizable fragment (Fc fragment) of immunoglobulin G (IgG) to form deposits that lodge in the joints and tissues.

Rheumatoid factor is present in patients with rheumatoid arthritis, but may also occur in patients with other autoimmune conditions such as systemic lupus erythematosus (SLE), Sjögren's syndrome, and occasionally scleroderma and polymyositis. It is also seen in the rheumatoid arthritis overlap syndromes, such as RA/SLE overlap and Scleroderma/RA overlap. The RF test may also yield a positive result in other conditions as well as in the absence of disease, especially with advancing age. Other conditions that may cause a positive RF test result include chronic active hepatitis, sarcoidosis, chronic infection, various cancers, and syphilis.

Autoantibodies directed against citrullinated proteins (e.g., anti-CCP [cyclic citrullinated peptide] antibodies) are specific serological markers for rheumatoid arthritis. Anti-CCP antibodies may be detected in roughly 50-60% of patients with early rheumatoid arthritis at “baseline” (at their initial encounter with a specialist, usually after 3-6 months of symptoms). See, e.g., Nell, V., et al. Arthritis Res. Ther. 5 (Suppl 1):16 (2003). The specificity of anti-CCP is around 95-98% in regards to undifferentiated forms of arthritis that do not develop into RA. IgM RF are often found in the same patients, but with much lower specificity for RA. One study using an anti-CCP assay showed a sensitivity of 55% and a specificity of 97% specificity for RA, when both anti-CCP and IgM RF were positive in the early stage of arthritis. See, e.g., Jansen, A. L., et al., J. Rheumatol. 29:2074-6 (2002). Another study showed even higher prevalence at the first visit to clinics—anti-CCP antibodies were found in 70% of such patients. Interestingly, using stored samples, anti-CCP could be detected 1.5 to 9 years before the onset of arthritis. See, e.g., Rantapää-Dahlqvist, S., et al., Arthritis Rheum. 48:2741-9 (2003). A study using an anti-CCP assay found progression from undifferentiated polyarthritis to RA in 93% of anti-CCP positive patients but only in 25% of anti-CCP negative patients after 3 years of follow up. See, e.g., van Gaalen, F. A., et al., Arthritis Res. Ther. 5 (suppl 1):28 (2003). Several observations have indicated that an anti-CCP positive result in early RA patients may develop a more erosive disease than those without anti-CCP.

### SUMMARY OF THE DISCLOSURE

It has now been discovered that the presence or absence of rheumatoid arthritis (RA) in a human subject, or the stage of rheumatoid arthritis in a subject afflicted with rheumatoid arthritis, or all of these, can be determined by an analysis of a biological sample from the subject for antibodies against a panel of antigens that does not necessarily include rheumatoid factor, or CCP or both. In many cases, however, the panel will include CCP and one, two, three, four or at least five (e.g., 5, 6, 7, 8, 9, 10, or all) other antigens selected from a specified list. In certain embodiments of the invention, the specified list is:
(i) BRAF1 506-525 (SEQ ID NO:1),
(ii) BRAF2 656-675 (SEQ ID NO:2),
(iii) Vimentin (protein) citrullinated (SEQ ID NO:3),
(iv) Vimentin 415-433 cit cyclic (SEQ ID NO:4), or an cyclic citrullinated variant binding RA autoantibodies;
(v) Vimentin 58-77 cit3 cyclic (SEQ ID NO:5), or an cyclic citrullinated variant binding RA autoantibodies;
(vi) Clusterin 231-250 cit sm1 cyclic (SEQ ID NO:6), or an cyclic citrullinated variant binding RA autoantibodies;
(vii) Fibrinogen A 556-575 cit sm cyclic (SEQ ID NO:7), or an cyclic citrullinated variant binding RA autoantibodies;
(viii) Fibrinogen A 616-635 cit sm cyclic (SEQ ID NO:8), or an cyclic citrullinated variant binding RA autoantibodies;
(ix) Histones2A H2A/a 1-20 cit sm2 cyclic (SEQ ID NO:9), or an cyclic citrullinated variant binding RA autoantibodies;
(x) Filaggrin 48-65 cit2v1 cyclic (SEQ ID NO:10), or an cyclic citrullinated variant binding RA autoantibodies; and
(xi) BRAF (catalytic domain from v raf murine sarcoma viral oncogene homologue B1, amino acids 416-766) (SEQ ID NO:11).

In some embodiments, the antigens in the panel are the peptides specified above, e.g., the biological sample is contacted to cyclic citrullinated peptide and the at least five of the peptides specified above.

Alternatively, in some embodiments, the biological sample is contacted to a plurality of peptides having at least one epitope of each of cyclic citrullinated peptide and the at least five members. Said another way, it will be appreciated the panel can alternatively comprise additional amino acids or fewer amino acids than specified so long as the peptide comprises the epitopes specified above. Thus, in some embodiments, the antigens further comprise one or more amino acids that are heterologous to the antigens, i.e., are not from the same naturally-occurring protein. For example, in some embodiments, the specified peptides will be linked to a linker amino acid sequence or an amino acid sequence that does not otherwise naturally occur adjacent to the antigen in native proteins. In some embodiments, the antigens can comprise one or more (e.g., 1, 2, 3, 4, 5, 6, 7, 8, 10 or more) additional native (not heterologous) amino acids from the naturally-occurring protein (for example, a peptide comprising BRAF1 500-530 comprises the specified antigen BRAF1 506-525). In other embodiments, the antigen can contain 1, 2, 3, 4, 5, 6, 7, 8, 9, or more fewer amino acids than specified above so long as at least one target-specific antigen remains in the peptide. As an example, instead of BRAF1 506-525, BRAF1507-525 might be used.

A number of the antigens listed above (i.e., iii-x) are citrullinated (“cit”, “cit3”, etc.), i.e., comprise one or more (e.g., 2, 3, 4, 5, 6, 7, 8, or more) citrullenes in place of a native arginine. As shown in the “SEQUENCES” section below, various arginines can be replaced with citrullenes. The antigens will comprise at least one citrullene but can comprise more than one.

Further, a number of the antigens (iv-x) are indicated as cyclic. The peptides are rendered cyclic by the addition of a pair of cysteines positioned within the native peptide sequence. In some embodiments, the cysteines are places at either end of the peptide. This option is exemplified in SEQ ID NO:4 below where a “C” is at either end of the sequence. Alternatively, one or both cysteine can be placed at an internal (not end) position of the peptide. For example, SEQ ID NO:5 has both introduced cysteines at an internal position. In some embodiments, the cysteines can be positioned to span a citrullene in the peptide sequence. One or both cysteines can be, in some embodiments, within 3, 4, 5, or 6 amino acids from the citrullene. While specific placement of cysteines to form cyclic peptides are shown in SEQ ID NO:s4-10, it will be appreciated that other placements of cysteine pairs is contemplated for each native peptide sequence (the native sequence being the sequences in any of SEQ ID NOs: 4, 5, 6, 7, 8, 9, or 10 lacking the cysteines.), thereby allowing for different cyclic options while presenting the same, or substantially the same, antigen/epitopes. Such options are considered “cyclic citrullinated variant binding RA autoantibodies” as used above.

With certain panels, the determinations of the presence or stage of rheumatoid arthritis can be made on subjects who have tested negative for CCP, i.e., for antibodies to CCP, and accordingly, CCP can be omitted from the panel.

Regardless of the composition of the panel, the results obtained can be correlated to the presence, absence, or stage of rheumatoid arthritis in the subject by comparison of the result for each panel member with a cutoff value for that panel member, and assessing the collective results for all panel members with the use of a readily obtained algorithm to define whether a given sample is “positive” or “negative.” The cutoff values are readily determined from samples representing subjects known to be afflicted with rheumatoid arthritis, or those known not to be afflicted with rheumatoid arthritis, or those known to be afflicted with rheumatoid arthritis and whose stage of rheumatoid arthritis is known.

Determinations in accordance with this invention can be made by incubating the sample with molecules of the panel of antigens and detecting whether any of the antigen molecules have become immunologically bound to antibodies from the sample. The antigen molecules with which the sample is incubated in this incubation step will be exogenous molecules of antigens, i.e., antigen molecules supplied externally and not those present in the sample if indeed any such molecules are present in the sample. These exogenous molecules can be identical copies of those in the subject that have generated the antibodies being detected, or they can be portions or segments of the antigen molecules in the subject, or molecules that bind immunologically with the antibodies with the same specificity and binding affinity. In certain cases, determinations of the particular exogenous antigen molecules that have become bound in this incubation step can be made, as well as of how many or to what degree these antigen molecules have become bound. In certain embodiments of the invention, therefore, the antibodies present in the sample are identified in terms of the antigens to which they have become bound in the incubation step. One means of such identification is to utilize antigen molecules that are immobilized on solid supports, with a different solid support for each antigen, i.e., with molecules of only one antigen being immobilized on any individual solid support, and with solid supports for different antigens being distinguishable from each other by means other than the antigens themselves. This can be achieved by using differentiation parameters associated with the solid supports, all supports bearing any one antigen be differentiable from all supports bearing any of the other antigens by the differentiation parameters.

The detection of immunological binding between antibodies from the sample and antigen molecules with which the sample is incubated can be achieved by the use of labeled anti-human antibodies. The antigen-antibody complexes formed by the incubation of the sample with the antigens in the panel can be themselves incubated with the labeled antibodies in a second incubation step. The label can then be detected and, if desired, quantified. Differentiation of the labels on the basis of the antibodies in the sample to which the labeled antibodies have become bound, i.e., determining which antibodies from the sample the detected labels are associated with, can then be achieved by the differentiation parameters mentioned above. The detected labels are thus correlated with the differentiation parameters. Correlation in this context refers to associating the label detection with the antibodies from the sample that the labels have become bound to (through the anti-human antibodies).

If the presence of rheumatoid arthritis, or a particular stage of the disease is detected, the methods can further comprise prescribing, counseling, or performing administration of one or more medical treatment, including but not limited to administration of one or more drug, for treating or ameliorating the disease.

The invention further resides in a kit for determining whether a human subject is afflicted with rheumatoid arthritis or for determining the stage that a human subject suffering from rheumatoid arthritis is in. The kit includes at least one antigen from the above list and in some embodiments a panel of antigens, each of which is immobilized on a solid support. The panel can include, e.g., any of the various antigens presented above (e.g., CCP and one, two, three, four or at least five (e.g., 5, 6, 7, 8, 9, 10, or all) other antigens), and the solid supports further contain differentiation parameters that are selected such that all of the supports bearing any one antigen of the panel are differentiable by these parameters from all of the supports bearing other antigens of the panel.

### DEFINITIONS

The term “label” or “detectable moiety” is used herein to denote a composition detectable by spectroscopic, photochemical, biochemical, immunochemical, chemical, or other physical means. Examples of labels are 32P, fluorescent dyes, electron-dense reagents, enzymes (e.g., as commonly used in an ELISA), biotin, digoxigenin, and haptens and proteins or other entities which can be made detectable, e.g., by incorporating a radiolabel into the peptide or by being used to detect antibodies specifically reactive with the peptide. The labels can be incorporated, for example, into antibodies and/or other proteins at any position. Any method known in the art for conjugating the antibody to the label can be employed, for example, using methods described in Hermanson, Bioconjugate Techniques 1996, Academic Press, Inc., San Diego. Alternatively, methods using high affinity interactions can achieve the same results where one of a pair of binding partners binds to the other, e.g., biotin and streptavidin. The proteins of the invention as described herein can be directly labeled as with isotopes, chromophores, lumiphores, chromogens, or indirectly labeled such as with biotin to which streptavidin in a complex with a fluorescent, radioactive, or other moiety that can be directly detected can then bind. Thus, a biotinylated antibody is considered a “labeled antibody” as used herein.

The term “antibody” as used herein refers to a polypeptide encoded by an immunoglobulin gene or immunoglobulin genes, or fragments thereof, which specifically bind and recognize an analyte (antigen). The recognized immunoglobulin light chains are classified as either kappa or lambda. Immunoglobulin heavy chains are classified as gamma, mu, alpha, delta, or epsilon, which in turn define the immunoglobulin classes, IgG, IgM, IgA, IgD and IgE, respectively.

An example of a structural unit of immunoglobulin G (IgG antibody) is a tetramer. Each such tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms “variable light chain” (VL) and “variable heavy chain” (VH) refer to these light and heavy chains, respectively.

Antibodies exist as intact immunoglobulins or as well-characterized fragments produced by digestion of intact immunoglobulins with various peptidases. Thus, for example, pepsin digests an antibody near the disulfide linkages in the hinge region to produce F(ab′)2, a dimer of Fab which itself is a light chain joined to VH-CH1 by a disulfide bond. The F(ab′)2 dimer can be reduced under mild conditions to break the disulfide linkage in the hinge region, thereby converting the F(ab′)2 dimer into two Fab′ monomers. The Fab′ monomer is essentially an Fab with part of the hinge region (see, Paul (Ed.), Fundamental Immunology, Third Edition, Raven Press, NY (1993)). While various antibody fragments are defined in terms of the digestion of an intact antibody, one of skill will appreciate that such fragments may be synthesized de novo either chemically or by utilizing recombinant DNA methodology. Thus, the term “antibody,” as used herein, also includes antibody fragments either produced by the modification of whole antibodies or by de novo synthesis using recombinant DNA methodologies such as single chain Fv.

The expression “specifically (or selectively)” in reference to binding to an antibody, or “specifically (or selectively) immunoreactive with” or “having binding specificity for,” when referring to a protein, peptide, or antigen, refers to a binding reaction which is determinative of the presence of the protein, peptide, or antigen in the presence of a heterogeneous population of proteins and other biologics. Thus, under designated immunoassay conditions, the specified antibodies bind to a particular protein and do not bind in a significant amount to other proteins present in the sample. Specific binding to an antibody under such conditions may require an antibody that is selected for its specificity for a particular protein. For example, antibodies raised against a protein can be selected to obtain antibodies specifically immunoreactive with that protein and not with other proteins. A variety of immunoassay formats may be used to select antibodies specifically immunoreactive with a particular protein. For example, solid-phase ELISA immunoassays, Western blots, or immunohistochemistry are routinely used to select monoclonal antibodies specifically immunoreactive with a protein. See, Harlow and Lane Antibodies, A Laboratory Manual, Cold Spring Harbor Publications, NY (1988) for a description of immunoassay formats and conditions that can be used to determine specific immunoreactivity. Typically, a specific or selective reaction will be at least twice the background signal or noise, and more typically more than 10 to 100 times background.

Antibodies for use in certain embodiments of the present invention are anti-human antibodies, particularly those anti-human antibodies that are labeled. Preferred among these anti-human antibodies are those that are antibodies to human IgG, those that are antibodies to human IgM, and those that are antibodies to human IgA.

The term “biological sample” encompasses a variety of sample types obtained from an organism. The term encompasses bodily fluids such as blood, saliva, serum, plasma, urine and other liquid samples of biological origin, solid tissue samples, such as a biopsy specimen or tissue cultures or cells derived therefrom and the progeny thereof. As described herein, typically, the biological sample will be a bodily fluid or tissue that contains detectable amounts of antibodies. The term encompasses samples that have been manipulated in any way after their procurement, such as by treatment with reagents, solubilization, sedimentation, or enrichment for certain components. The term encompasses a clinical sample, and also includes cells in cell culture, cell supernatants, cell lysates, serum, plasma, other biological fluids, and tissue samples. Preferred biological samples are blood samples, plasma samples, and serum samples.

The term “solid support” is used herein to denote a solid inert surface or body to which an agent, such as an antibody or an antigen, that is reactive in any of the binding reactions described herein can be immobilized. The term “immobilized” as used herein denotes a molecularly based coupling that is not dislodged or de-coupled under any of the conditions imposed during any of the steps of the assays described herein. Such immobilization can be achieved through a covalent bond, an ionic bond, an affinity-type bond, or any other chemical bond.

The term “particles” is used herein to denote solid bodies, often with linear dimensions on the micron scale (i.e., less than 100 microns), of any shape or surface texture. The term “beads” is herein to denote particles that are spherical or near-spherical in shape, often polymeric in composition.

“Multiplex” assays are analyses that simultaneously measure the levels of more than one analyte in a single sample.

The term “endogenous” as used herein refers to molecules or agents that are introduced into assay media of the present invention from sources other than those of the human subject from which the biological sample has been drawn. Endogenous molecules or agents include those that are identical to molecules or agents present in the body of the subject and those that are not.

### DESCRIPTION OF SELECTED EMBODIMENTS

While various panels of antigens drawn from the list above in accordance with the criteria set forth above can be used in the assays described herein, the effectiveness of any panel can be determined by comparing the results obtained with the panel to those obtained with conventional tests for RA and determining whether there is agreement between the two results, and particularly whether the assays performed within the scope of this invention are superior. Superiority is this context means that the number of false positives, false negatives, or both is reduced, and thus that the assay provides greater specificity. Any of various algorithms that will be readily apparent to those of skill in the art can be used. In most cases, one will want to determine a cutoff value for each antigen (“marker”) to use in the algorithm, and one example of a means for assigning a cutoff value for a single marker is to study samples from a substantial number of healthy adult human subjects, such as 50 or more, or perhaps 50 to 500, and identify the level of that antigen at the 95^{th} percentile, or perhaps the 98^{th} percentile, as the cutoff value. Samples from test subjects (i.e., those whose presence or absence of RA is to be determined) are then analyzed for the individual markers, and an algorithm to analyze the results is applied. The algorithm will be one that determines which samples will be deemed “positive,” the remainder being deemed “negative.” According to one example of such an algorithm, “positive” samples will be those in which any of the following three criteria are met:
(1) The value for the citrullinated vimentin marker is equal to or greater than 10.0 times the cutoff value.
(2) The value for any citrullinated marker other than citrullinated vimentin is equal to or greater than 5.0 times the cutoff value for that marker.
(3) The value of the CCP marker or the BRAF marker is equal to or greater than 2.0 times the cutoff value for that marker.
(4) The value of CCP marker is equal to or greater than 0.5 times the CCP cutoff value and the value of any other marker is equal to or greater than 2.0 times the cutoff value.

The effectiveness, and improvement where desired, of the use of marker panels within the scope of this invention can be determined by comparing the results of any marker panel within the scope of this invention on one set of known samples with the results obtained by an ELISA-based assay for anti-CCP, for example, on the same set of samples. The most desirable marker panels will be those in which the number of false negatives are at least 5% less than one would obtain with an ELISA-based assay for anti-CCP, or perhaps at least 10% less, and perhaps further at least 10% less. In absolute terms, the number of false negatives in the most desirable panels within the scope of this invention will preferably be 30% or less, more preferably 20% or less, and most preferably 10% or less.

In embodiments of the invention that involve the use of labeled anti-human antibodies for purposes of detecting antigen-antibody binding, the labels can be any substance or component that directly or indirectly emits or generates a detectable signal. In some embodiments, the labels are fluorophores, many of which are reported in the literature and thus known to those skilled in the art, and many of which are readily available from commercial suppliers to the biotechnology industry. Literature sources for fluorophores include Cardullo et al., Proc. Natl. Acad. Sci. USA 85: 8790-8794 (1988); Dexter, D. L., J. of Chemical Physics 21: 836- 850 (1953); Hochstrasser et al., Biophysical Chemistry 45: 133-141 (1992); Selvin, P., Methods in Enzymology 246: 300-334 (1995); Steinberg, I., Ann. Rev. Biochem., 40: 83-114 (1971); Stryer, L., Ann. Rev. Biochem. 47: 819-846 (1978); Wang et al., Tetrahedron Letters 31: 6493-6496 (1990); and Wang et al., Anal. Chem. 67: 1197-1203 (1995).

The following are examples of fluorophores that can be used as labels:
4-acetamido-4′-isothiocyanatostilbene-2,2′disulfonic acid acridine
acridine isothiocyanate
5-(2′-aminoethyl)aminonaphthalene-1-sulfonic acid (EDANS)
4-amino-N-[3-vinylsulfonyl)phenyl]naphthalimide-3,5disulfonate
N-(4-anilino-1-naphthyl)maleimide
anthranilamide
BODIPY
Brilliant Yellow
coumarin
7-amino-4-methylcoumarin (AMC, Coumarin 120)
7-amino-4-trifluoromethylcoumarin (Coumaran 151)
cyanine dyes
cyanosine
4′,6-diaminidino-2-phenylindole (DAPI)
5′,5″-dibromopyrogallol-sulfonaphthalein (Bromopyrogallol Red)
7-diethylamino-3-(4′-isothiocyanatophenyl)-4-methylcoumarin
diethylenetriamine pentaacetate
4,4′-diisothiocyanatodihydro-stilbene-2,2′-disulfonic acid
4,4′-diisothiocyanatostilbene-2,2′-disulfonic acid
5-[dimethylamino]naphthalene-1-sulfonyl chloride (DNS, dansylchloride)
4-(4′-dimethylaminophenylazo)benzoic acid (DABCYL)
4-dimethylaminophenylazophenyl-4′-isothiocyanate (DABITC)
eosin
eosin isothiocyanate
erythrosin B
erythrosin isothiocyanate
ethidium
5-carboxyfluorescein (FAM)
5-(4,6-dichlorotriazin-2-yl)aminofluorescein (DTAF)
2′,7′-dimethoxy-4′5′-dichloro-6-carboxyfluorescein (JOE)
fluorescein
fluorescein isothiocyanate
fluorescamine
IR144

IR1446
Malachite Green isothiocyanate
4-methylumbelliferone
ortho cresolphthalein
nitrotyrosine
pararosaniline
Phenol Red
phycoerythrin (including but not limited to B and R types)
o-phthaldialdehyde
pyrene
pyrene butyrate
succinimidyl 1-pyrene butyrate
quantum dots
Reactive Red 4 (Cibacron™ Brilliant Red 3B-A)
6-carboxy-X-rhodamine (ROX)
6-carboxyrhodamine (R6G)
lissamine rhodamine B sulfonyl chloride rhodamine
rhodamine B
rhodamine 123
rhodamine X isothiocyanate
sulforhodamine B
sulforhodamine 101
sulfonyl chloride derivative of sulforhodamine 101 (Texas Red)
N,N,N′,N′-tetramethyl-6-carboxyrhodamine (TAMRA)
tetramethyl rhodamine
tetramethyl rhodamine isothiocyanate (TRITC)
riboflavin
rosolic acid
lanthanide chelate derivatives

A prominent group of fluorophores for immunoassays are fluorescein, fluorescein isothiocyanate, phycoerythrin, rhodamine B, and Texas Red (sulfonyl chloride derivative of sulforhodamine 101). Phycoerythrin is particularly prominent. Any of the fluorophores in the list preceding this paragraph can be attached to anti-human antibodies by conventional covalent bonding, using appropriate functional groups on the fluorophores and on the antibodies. The recognition of such groups and the reactions to form the linkages will be readily apparent to those skilled in the art.

Other labels that can be used in place of the fluorophores are radioactive labels and enzyme labels. These are likewise known in the art.

The determination that immunological binding has occurred constitutes one or more steps in certain embodiments of this invention, and this can involve the separation or recovery of antigen-antibody complexes from unbound antigen or antibody. One means of achieving such separation or recovery is by the use of solid supports, particularly for the antigens with which the biological sample is incubated in the first immunological binding reaction.

Any type of solid support can be used in the invention. The solid support can be the wall or floor of an assay vessel, or a dipstick or other implement to be inserted into an assay vessel, or particles placed inside or suspended in an assay vessel. Particles, and especially beads, are particularly useful in many embodiments, including beads that are microscopic in size (i.e., microparticles) and formed of a polymeric material. Polymers useful as microparticles are those that are chemically inert relative to the components of the biological sample and to the assay reagents other than the binding members that are immobilized on the microparticle surface. Preferred microparticle materials, particularly when fluorescent labels are used in the assay, are those with minimal autofluorescence, and that are solid and insoluble in the sample and in any buffers, solvents, carriers, diluents, or suspending agents used in the assay, in addition to allowing immobilization of the assay reagent. Examples of suitable polymers are polystyrenes, polyesters, polyethers, polyolefins, polyalkylene oxides, polyamides, polyurethanes, polysaccharides, celluloses, and polyisoprenes. Crosslinking is useful in many polymers for imparting structural integrity and rigidity to the microparticle. The size range of the microparticles can vary. In some embodiments, the microparticles range in diameter from about 0.3 micrometers to about 100 micrometers, and other embodiments, from about 0.5 micrometers to about 40 micrometers, and in still other embodiments, from about 2 micrometers to about 10 micrometers.

Particle recovery and washing can be facilitated by the use of particles that are formed of or contain a magnetically responsive material, i.e., any material that responds to a magnetic field. Separation of the solid and liquid phases, either after incubation or after a washing step, is then achieved by imposing a magnetic field on the reaction vessel in which the particles and sample are incubated, causing the particles to adhere to the wall of the vessel and thereby permitting the liquid to be removed by decantation or aspiration. Magnetically responsive materials of interest in this invention include paramagnetic materials, ferromagnetic materials, ferrimagnetic materials, and metamagnetic materials. Examples, include, e.g., iron, nickel, and cobalt, as well as metal oxides such as Fe₃O₄, BaFe₁₂O₁₉, CoO, NiO, Mn₂O₃, Cr₂O₃, and CoMnP.

Methods of, and instrumentation for, applying and removing a magnetic field as part of an assay are known to those skilled in the art and reported in the literature. Examples of literature reports are Forrest et al., U.S. Pat. No. 4,141,687 (Technicon Instruments Corporation, Feb. 27, 1979); Ithakissios, U.S. Pat. No. 4,115,534 (Minnesota Mining and Manufacturing Company, Sep. 19, 1978); Vlieger, A. M., et al., Analytical Biochemistry 205:1-7 (1992); Dudley, Journal of Clinical Immunoassay 14:77-82 (1991); and Smart, Journal of Clinical Immunoassay 15:246-251 (1992).

Magnetically responsive material can be dispersed throughout the polymer, applied as a coating on the polymer surface or as one of two or more coatings on the surface, or incorporated or affixed in any other manner that secures the material in to the particle. The quantity of magnetically responsive material in the particle is not critical and can vary over a wide range. The quantity can affect the density of the microparticle, however, and both the quantity and the particle size can affect the ease of maintaining the microparticle in suspension for purposes of achieving maximal contact between the liquid and solid phase and for facilitating flow cytometry. An excessive quantity of magnetically responsive material in the microparticles may produce autofluorescence at a level high enough to interfere with the assay results. Therefore, in some embodiments, the concentration of magnetically responsive material is low enough to minimize any autofluorescence emanating from the material. With these considerations in mind, the magnetically responsive material in a particle in accordance with this invention is, for example, from about 0.05% to about 75% by weight of the particle as a whole. In some embodiments, the weight percent range is from about 1% to about 50%, e.g., from about 2% to about 25%, e.g., from about 2% to about 8%.

Coating of the particle surface with the appropriate assay reagent can be achieved by electrostatic attraction, specific affinity interaction, hydrophobic interaction, or covalent bonding. The polymer can be derivatized with functional groups for covalent attachment of the assay reagents by conventional means, notably by the use of monomers that contain the functional groups, such monomers serving either as the sole monomer or as a co-monomer. Examples of suitable functional groups are amine groups (—NH₂), ammonium groups (—NH₃⁺ or —NR₃⁺), hydroxyl groups (—OH), carboxylic acid groups (—COOH), and isocyanate groups (—NCO). Useful monomers for introducing carboxylic acid groups into polyolefins, for example, are acrylic acid and methacrylic acid.

Linking groups can be used as a means of increasing the density of reactive groups on the particle surface and also as a means of decreasing steric hindrance Linking groups can also be used as a means of securing coating materials to the particle surfaces. Certain linking groups are monofunctional linkers comprising a reactive group as well as multifunctional crosslinkers comprising two or more reactive groups capable of forming a bond with two or more different functional targets (e.g., peptides, proteins, macromolecules, semiconductor nanocrystals, or substrate). In some embodiments, the multifunctional crosslinkers are heterobifunctional crosslinkers comprising two different reactive groups. Examples of suitable reactive groups are thiol (—SH), carboxylate (—COOR), carboxyl (—COOH), carbonyl (—C(O)—), amine (NH₂), hydroxyl (—OH), aldehyde (—CHO), hydroxyl (—OH), active hydrogen, ester, phosphate (—PO₃), and photoreactive moieties. Examples of amine reactive groups are isothiocyanates, isocyanates, acyl azides, NHS esters, sulfonyl chlorides, aldehydes and glyoxals, epoxides and oxiranes, carbonates, arylating agents, imidoesters, carbodiimides, and anhydrides. Examples of thiol-reactive groups are haloacetyl and alkyl halide derivates, maleimides, aziridines, acryloyl derivatives, arylating agents, and thiol-disulfides exchange reagents. Examples of carboxylate reactive groups are diazoalkanes and diazoacetyl compounds, such as carbonyldiimidazoles and carbodiimides. Examples of hydroxyl reactive groups are epoxides and oxiranes, carbonyldiimidazole, oxidation with periodate, N,N′-disuccinimidyl carbonate or N-hydroxylsuccimidyl chloroformate, enzymatic oxidation, alkyl halogens, and isocyanates. Examples of aldehyde and ketone reactive groups are hydrazine derivatives for Schiff base formation or reduction amination. Examples of active hydrogen reactive groups are diazonium derivatives for Mannich condensation and iodination reactions. Examples of photoreactive groups are aryl azides and halogenated aryl azides, benzophenones, diazo compounds, and diazirine derivatives.

Other suitable reactive groups and classes of reactions useful in practicing the present invention are generally those that are well known in the art of bioconjugate chemistry. Currently favored classes of reactions available with reactive chelates are those which proceed under relatively mild conditions. These include, but are not limited to, nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, Advanced Organic Chemistry, 3rd Ed., John Wiley & Sons, New York, 1985; Hermanson, Bioconjugate Techniques, Academic Press, San Diego, 1996; and Feeney et al., Modification Of Proteins; Advances in Chemistry Series, Vol. 198, American Chemical Society, Washington, D.C., 1982.

In some embodiments, the functional group is a heterobifunctional crosslinker comprising two different reactive groups that contain heterocyclic rings that can interact with peptides and proteins. For example, heterobifunctional crosslinkers such as N-[γ-maleimidobutyryloxy]succinimide ester (GMBS) or succinimidyl 4-[N-maleimidomethyl]cyclohexane-1-carboxylate (SMCC) comprise an amine reactive group and a thiol-reactive group that can interact with amino and thiol groups within peptides or proteins. Additional combinations of reactive groups suitable for heterobifunctional crosslinkers include, for example, carbonyl and sulfhydryl reactive groups; amine and photoreactive groups; sulfhydryl and photoreactive groups; carbonyl and photoreactive groups; carboxylate and photoreactive groups; and arginine and photoreactive groups. Examples of suitable useful linking groups are polylysine, polyaspartic acid, polyglutamic acid and polyarginine. N-hydroxysuccinimide (NHS), CMC 1-cyclohexyl-3-(2-morpholinoethyl)carbodiimide (CMC), N-Hydroxybenzotriazole (HOBt), and/or other crosslinking agents may be used.

Particles formed by conventional emulsion polymerization techniques from a wide variety of starting monomers are favorable in many cases since they exhibit at most a low level of autofluorescence. Conversely, particles that have been modified to increase their porosity and hence their surface area, i.e., those particles that are referred to in the literature as “macroporous” particles, tend to exhibit high autofluorescence and are often less desirable. Autofluorescence increases with increasing size and increasing amounts of divinylbenzene monomer.

Multiplexing, i.e., the performance of simultaneous assays for all antibodies for all antigens in a given panel, can be performed with the use of solid supports by utilization of differentiation parameters, as mentioned above.

One example of a differentiation parameter is the particle diameter, where the solid supports are particles divided into groups with nonoverlapping diameter subranges. The widths of the diameter subranges and the spacing between mean diameters of adjacent subranges in these embodiments are selected to permit differentiation of the subranges by flow cytometry, and such selection will be readily apparent to those skilled in the use of and instrumentation for flow cytometry. In this specification, the term “mean diameter” refers to a number average diameter.

In some embodiments, the subrange width is about ±5% CV or less of the mean diameter, where “CV” stands for “coefficient of variation” and is defined as the standard deviation of the particle diameter divided by the mean particle diameter, times 100 percent. The minimum spacing between mean diameters among the various subranges can vary depending on the microparticle size distribution, the ease of segregating microparticles by size for purposes of attaching different assay reagents, and the type and sensitivity of the flow cytometry equipment. In some embodiments, best results will be achieved when the mean diameters of different subranges are spaced apart by at least about 6% of the mean diameter of one of the subranges, e.g., at least about 8% of the mean diameter of one of the subranges, e.g., at least about 10% of the mean diameter of one of the subranges. In some embodiments, the standard deviation of the particle diameters within each subrange is less than one third of the separation of the mean diameters of adjacent subranges.

Another example of a differentiation parameter that can be used to distinguish among different groups of particles is fluorescence. Differentiation by fluorescence is accomplished by incorporating one or more fluorescent materials in the particles, the fluorescent materials having different fluorescent emission spectra and being distinguishable on this basis. Differentiation can be achieved by using fluorescent materials that have different fluorescence intensities or that emit fluorescence at different wavelengths, or by varying the amount of fluorescent material incorporated. Differentiation by fluorophores can also be achieved by using combinations of fluorophores for each particle subgroup. For example, the particle can be made to contain a red fluorochrome such as Cy5 together with a far-red fluorochrome such as Cy5.5, at different relative amounts for different subgroups. Additional fluorochromes can be used to further expand the system. Each microparticle can thus contain a plurality of fluorescent dyes at varying wavelengths.

By using fluorescence emissions at different wavelengths, the wavelength difference can be used to distinguish the particle groups from each other, while also distinguishing the labels in the labeled anti-human antibodies from the labels that differentiate one particle group from another. An example of a fluorescent substance that can be used as a means of distinguishing particle groups is fluorescein and an example of a substance that can be used for the assay detection is phycoerythrin. In the use of this example, different particle groups can be dyed with differing concentrations of fluorescein to distinguish them from each other, while phycoerythrin is used as the label on the various labeled binding members used in the assay.

Another example of a differentiation parameter that can be used to distinguish among the various groups of particles is light scatter. Side angle light scatter varies with particle size, granularity, absorbance and surface roughness, while forward angle light scatter is mainly affected by size and refractive index. Varying any of these qualities can result in light scatter differences that can serve as a means of distinguishing the various groups.

Still another example of a differentiation parameter is absorbance. When light is applied to particles, the absorbance of the light by the particles is indicated mostly by a change in the strength of the laterally (side-angle) scattered light while the strength of the forward-scattered light is relatively unaffected. Consequently, the difference in absorbance between various colored dyes associated with the particles is determined by observing differences in the strength of the laterally scattered light.

A still further example of a differentiation parameter is the number of particles in each group. When the number of particles in each group is varied in a known way, the count of particles having various assay responses can be associated with a particular assay by the number of particles having each response.

As the above examples illustrate, a wide array of parameters or characteristics can be used as differentiation parameters to distinguish the particles of one group from those of another. The differentiation parameters may arise from particle size, from particle composition, from particle physical characteristics that affect light scattering, from excitable fluorescent dyes or colored dyes that impart different emission spectra and/or scattering characteristics to the particles, or from different concentrations of one or more fluorescent dyes. When the distinguishable particle parameter is a fluorescent dye or color, it can be coated on the surface of the particle, embedded in the particle, or bound to the molecules of the particle material. Thus, fluorescent particles can be manufactured by combining the polymer material with the fluorescent dye, or by impregnating the particle with the dye. Particles with dyes already incorporated and thereby suitable for use in the present invention are commercially available, from suppliers such as Spherotech, Inc. (Libertyville, Ill., USA) and Molecular Probes, Inc. (Eugene, Oreg., USA).

When particles are used, particularly microparticles, the use of flow cytometry is a convenient way of sorting the particles by the differentiation parameter, and also in many cases of determining whether a label has been attached to the particle through the assay components as a result of the assay reaction.

Methods of, and instrumentation for, flow cytometry are known in the art, and can be used in the practice of the present invention. Flow cytometry in general resides in the passage of a suspension of particles (or cells) in as a stream through a light beam and coupled to electro-optical sensors, in such a manner that only one particle at a time passes the region of the sensors. As each particle passes this region, the light beam is perturbed by the presence of the particle, and the resulting scattered and fluoresced light are detected. The optical signals are used by the instrumentation to identify the subgroup to which each particle belongs, along with the presence and amount of label, so that individual assay results are achieved. Descriptions of instrumentation and methods for flow cytometry are found in the literature. Examples are McHugh, “Flow Microsphere Immunoassay for the Quantitative and Simultaneous Detection of Multiple Soluble Analytes,” Methods in Cell Biology 42, Part B (Academic Press, 1994); McHugh et al., “Microsphere-Based Fluorescence Immunoassays Using Flow Cytometry Instrumentation,” Clinical Flow Cytometry, Bauer, K. D., et al., eds. (Baltimore, Md., USA: Williams and Williams, 1993), pp. 535-544; Lindmo et al., “Immunometric Assay Using Mixtures of Two Particle Types of Different Affinity,” J. Immunol. Meth. 126: 183-189 (1990); McHugh, “Flow Cytometry and the Application of Microsphere-Based Fluorescence Immunoassays,” Immunochemica 5: 116 (1991); Horan et al., “Fluid Phase Particle Fluorescence Analysis: Rheumatoid Factor Specificity Evaluated by Laser Flow Cytophotometry,” Immunoassays in the Clinical Laboratory, 185-189 (Liss 1979); Wilson et al., “A New Microsphere-Based Immunofluorescence Assay Using Flow Cytometry,” J. Immunol. Meth. 107: 225-230 (1988); Fulwyler et al., “Flow Microsphere Immunoassay for the Quantitative and Simultaneous Detection of Multiple Soluble Analytes,” Meth. Cell Biol. 33: 613-629 (1990); Coulter Electronics Inc., United Kingdom Patent No. 1,561,042 (published Feb. 13, 1980); and Steinkamp et al., Review of Scientific Instruments 44(9): 1301-1310 (1973).

The methods of the present invention, and the kits of the present invention that contain materials for use in practicing the methods, allow for the simultaneous detection and optionally quantification of the various antibodies in a biological sample. The presence of these antibodies or of subgroups of these antibodies are associated can be an indication of the presence, absence, or stage of rheumatoid arthritis, and other autoimmune diseases as well, in the subject from whom the sample was taken. In some embodiments, the detection and/or quantification of some or all of the various antibodies in a sample is used to provide a prognosis or to assess the efficacy of a pharmaceutical (anti-arthritis drug, for example) treatment. Diagnosis, prognosis, or assessing pharmaceutical efficacy can be achieved for example by correlating the amounts of certain antibodies in the sample with known amounts associated with healthy individuals, diseased individuals, or both.

### EXAMPLE 1

This example illustrates an assay in accordance with the present invention performed on a set of 389 samples from adult human subjects that were already diagnosed with rheumatoid arthritis (RA) for the first time within the six months preceding the study, to determine the sensitivity of the assay. Cutoff values were established by performing the same test on a normal sample set of 168 samples, i.e., samples from individuals not exhibiting symptoms of RA. The studies were performed using standard LUMINEX® bead-based assay technology (Luminex Corporation, Austin, Tex., USA) involving incubating serum samples with beads to which antigens were attached, the antibodies being specific for each of a panel of serum autoantibodies to a panel of markers, followed by incubating the beads with phycoerythrin-labeled labeled anti-human IgG to detect the bound antibodies. Nine markers were used: CCP, Vimentin citrullinated (recombinant), Vimentin 58-77 cit3 cyclic, Vimentin 415-433 cit cyclic, Vimentin 58-77 cit3 sm1 cyclic, BRAF1 506-525, BRAF2 656-675, Histones2A/a 1-20 cit small-2 cyclic, and Fibrinogen A (616-635) cit3 small cyclic. All data was reported in relative fluorescence units (RFI). Three of the “normal” samples indicated the presence of RA and were excluded from the cutoff value determinations. Using the results from the remaining 165 normal samples, the cutoff value of each marker was selected as the 98^{th} percentile for that marker.

Sorting of the data was done by the following algorithm:
For the citrullinated vimentin marker, if any test sample has a value equal to or greater than 10.0 times the cutoff, the result was designated “positive.”
For all of the test samples (i.e., from the set of 389) in which the value of any citrullinated marker other than citrullinated vimentin, a result equal to or greater than 5.0 times the cutoff value for that marker was designated “positive.”
All test samples in which the CCP or BRAF marker value was equal to or greater than 2.0 times the cutoff value for that marker were designated “positive.”
All test samples in which the CCP value was equal to or greater than 0.5 times the CCP cutoff value and the value of any other marker was equal to or greater than 2.0 times the cutoff value were designated “positive.”

According to this algorithm, 254 of the 389 test samples were deemed “positive,” indicating a sensitivity of 65.3% for the assay. For comparison, the same 389 test samples were tested for anti-CCP using BIOPLEX™ 200 System of Bio-Rad Laboratories, Inc. (Hercules, Calif., USA). The BIOPLEX™ 200 tests revealed only 226 positive samples (58.1%).

### EXAMPLE 2

This examples illustrates an assay in accordance with the present invention, performed on serum samples from 323 adult human subjects, each having received a clinical diagnosis of RA, and the assay results were analyzed using cutoff values established from samples from 106 normal patients (showing no RA symptoms). All determinations were made using the same LUMINEX® bead-based assay technology used in Example 1. Nine markers were used: CCP, Vimentin citrullinated (recombinant), Vimentin 58-77 cit3 cyclic, Vimentin 415-433 cit cyclic, Vimentin 58-77 cit3 sm1 cyclic, BRAF1 506-525, BRAF2 656-675, Histones2A/a 1-20 cit small-2 cyclic, and Fibrinogen A (616-635) cit3 small cyclic. The results were analyzed by the same algorithm as that of Example 1. The 323 test samples from RA-diagnosed subjects were separately tested for anti—CCP according to a commercial ELISA kit (DIASTAT™ of Axis-Shield Diagnostics plc, Dundee, Scotland). The DIASTAT ELISA test on the 323 test samples showed 241 to be positive and 82 to be negative. All of the 241 test samples that were positive by the DIASTAT ELISA test were also positive by the test according to the present invention. Of the 82 samples that were negative by the DIASTAT ELISA test, twelve, or 15%, read positive by the test of the present invention, resulting in a sensitivity of 78.3% vs. 74.6% for the DIASTAT ELISA test.

To summarize Examples 1 and 2, the test method of the present invention in both examples yielded fewer false negatives—12% fewer in Example 1 and 15% fewer in Example 2. In both cases the number of false positives obtained with the test method of the present invention amounted to less than 2%.

In the claims appended hereto, the term “a” or “an” is intended to mean “one or more.” The term “comprise” and variations thereof such as “comprises” and “comprising,” when preceding the recitation of a step or an element, are intended to mean that the addition of further steps or elements is optional and not excluded. All patents, patent applications, and other published reference materials cited in this specification are hereby incorporated herein by reference in their entirety. Any discrepancy between any reference material cited herein or any prior art in general and an explicit teaching of this specification is intended to be resolved in favor of the teaching in this specification. This includes any discrepancy between an art-understood definition of a word or phrase and a definition explicitly provided in this specification of the same word or phrase.

### SEQUENCES

(i) BRAF1 506-525 (SEQ ID NO:1)

RKTRHVNILLFMGYSTKPQL

(ii) BRAF2 656-675 (SEQ ID NO:2)

YSNINNRDQIIFMVGRGYLS

(iii) Vimentin (protein) citrullinated (SEQ ID NO:3)

MSTRSVSSSSYRRMFGGPGTASRPSSSRSYVTTSTRTYSLGS
ALRPSTSRSLYASSPGGVYATRSSAVRLRSSVPGVRLLQDS
VDFSLADAINTEFKNTRTNEKVELQELNDRFANYIDKVRFL
EQQNKILLAELEQLKGQGKSRLGDLYEEEMRELRRQVDQL
TNDKARVEVERDNLAEDIMRLREKLQEEMLQREEAENTLQ
SFRQDVDNASLARLDLERKVESLQEEIAFLKKLHEEEIQELQ
AQIQEQHVQIDVDVSKPDLTAALRDVRQQYESVAAKNLQE
AEEWYKSKFADLSEAANRNNDALRQAKQESTEYRRQVQS
LTCEVDALKGTNESLERQMREMEENFAVEAANYQDTIGRL
QDEIQNMKEEMARHLREYQDLLNVKMALDIEIATYRKLLE
GEESRISLPLPNFSSLNLRETNLDSLPLVDTHSKRTLLIKTVE
TRDGQVINETSQHHDDLE

(iv) Vimentin 415-433 cit cyclic (SEQ ID NO:4)

CLPNFSSLNL[CIT]ETNLDSLPLC

This peptide is cyclic due to a disulfide bond between the first and last cysteine (C); [CIT]=Citrulline
(v) Vimentin 58-77 cit3 cyclic (SEQ ID NO:5)

GGCVYAT[R/CIT]SSACV[R/CIT]L[R/CIT]SSVPGV

This peptide is cyclic due to a disulfide bond between the bolded and underlined cysteines (C); [R/CIT]=Arginine or Citrulline, however, the peptide comprises at least one CIT; bolded and underlined options are those tested in the examples.
(vi) Clusterin 231-250 cit sm1 cyclic (SEQ ID NO:6)

CHFS[R/CIT]ASSCIDELFQD[R/CIT]FFT[R/CIT]

This peptide is cyclic due to a disulfide bond between the bolded and underlined cysteines (C); [R/CIT]=Arginine or Citrulline, however, the peptide comprises at least one CIT; bolded and underlined options are those tested in the examples.
(vii) Fibrinogen A 556-575 cit sm cyclic (SEQ ID NO:7)

NTKESSSHHPGCAEFPS[CIT]GKC

This peptide is cyclic due to a disulfide bond between the bolded and underlined cysteines (C); [CIT]=Citrulline
(viii) Fibrinogen A 616-635 cit sm cyclic (SEQ ID NO:8)

THSTK[R/CIT]CHAKS[R/CIT]PV[R/CIT]GIHTSC

This peptide is cyclic due to a disulfide bond between the bolded and underlined cysteines (C); [R/CIT]=Arginine or Citrulline, however, the peptide comprises at least one CIT; bolded and underlined options are those tested in the examples.
(ix) Histones2A H2A/a 1-20 cit sm2 cyclic (SEQ ID NO:9)

MSG[R/CIT]GKQGCKA[R/CIT]AKAKT[R/CIT]SSC

This peptide is cyclic due to a disulfide bond between the bolded and underlined cysteines (C); [R/CIT]=Arginine or Citrulline, however, the peptide comprises at least one CIT; bolded and underlined options are those tested in the examples.

(xii) Filaggrin 48-65 cit2v1 cyclic (SEQ ID NO:10)

CTIHAHPGS[R/CIT][R/CIT]GG[R/CIT]HGYHHC

This peptide is cyclic due to a disulfide bond between the bolded and underlined cysteines (C); [R/CIT]=Arginine or Citrulline, however, the peptide comprises at least one CIT; bolded and underlined options are those tested in the examples.
(xiii) BRAF (catalytic domain from v raf murine sarcoma viral oncogene homologue B1, amino acids 416-766) (SEQ ID NO:11)

LQKSPGPQRERKSSSSSEDRNRMKTLGRRDSSDDWEIPDGQ
ITVGQRIGSGSFGTVYKGKWHGDVAVKMLNVTAPTPQQLQ
AFKNEVGVLRKTRHVNILLFMGYSTKPQLAIVTQWCEGSSL
YHHLHIIETKFEMIKLIDIARQTAQGMDYLHAKSIIHRDLKS
NNIFLHEDLTVKIGDFGLATVKSRWSGSHQFEQLSGSILWM
APEVIRMQDKNPYSFQSDVYAFGIVLYELMTGQLPYSNINN
RDQIIFMVGRGYLSPDLSKVRSNCPKAMKRLMAECLKKKR
DERPLFPQILASIELLARSLPKIHRSASEPSLNRAGFQTEDFSL
YACASPKTPIQAGGYGAFPVH

What is claimed is:

1. A method for determining whether a human subject is afflicted with rheumatoid arthritis or a stage of affliction of a human subject so afflicted, said method comprising:
(a) detecting in a biological sample from said subject levels of antibodies that bind to antigens of a panel of antigens, wherein said panel comprises cyclic citrullinated peptide and at least five members selected from the group consisting of
(i) BRAF1 506-525,
(ii) BRAF2 656-675,
(iii) Vimentin (protein) citrullinated,
(iv) Vimentin 415-433 cit cyclic, and
(v) Vimentin 58-77 cit3 cyclic,
(vi) Clusterin 231-250 cit sm1 cyclic,
(vii) Fibrinogen A 556-575 cit sm cyclic,
(viii) Fibrinogen A 616-635 cit sm cyclic,
(ix) Histones2A H2A/a 1-20 cit sm2 cyclic
(x) Filaggrin 48-65 cit2v1 cyclic, and
(xi) BRAF (catalytic domain from v raf murine sarcoma viral oncogene homologue B1, amino acids 416-766) and
(b) correlating said levels so detected to the presence, absence, or stage of rheumatoid arthritis in said subject.

2. The method of claim 1, comprising contacting said biological sample to said panel.

3. The method of claim 1, comprising contacting said biological sample to a plurality of peptides having at least one epitope of each of cyclic citrullinated peptide and the at least five members.

4. The method of claim 1 wherein step (a) comprises:
(1) incubating said sample with a plurality of solid supports having molecules of said antigens immobilized thereon, each of said solid supports having molecules of only one of said antigens of said panel thereon and said solid supports bearing differentiation parameters selected such that all said supports bearing any one antigen of said panel are differentiable from all said supports bearing other antigens of said panel, and performing said incubation under conditions promoting immunological binding of said antibodies if present in said sample to said solid support-immobilized antigens;
(2) recovering said solid supports from said sample;
(3) incubating said solid supports so recovered with a solution of labeled anti-human antibody under conditions promoting binding of said antibodies if present on said solid supports to said labeled anti-human antibody;
(4) recovering said solid supports from said labeled anti-human antibody solution; and
(5) detecting label bound to said solid supports thus recovered from said labeled anti-human antibody solution and correlating said label so detected with said differentiation parameters to obtain values individually representative of levels of said antibodies in said sample.

5. The method of claim 4 wherein said solid supports are beads, step (1) comprises incubating said sample with a mixture of said beads in a first suspension comprising said beads and said sample, step (2) comprises recovering said beads from said first suspension, step (3) comprises incubating said beads in a second suspension comprising said beads and said solution of labeled anti-human antibody, and step (4) comprises recovering said beads from said second suspension.

6. The method of claim 5 wherein said differentiation parameters are differentiable by flow cytometry, and step (5) comprises detecting said label and sorting said beads by flow cytometry.

7. The method of claim 4 wherein said labeled anti-human antibody is labeled anti-human IgG.

8. The method of claim 4 wherein said labeled anti-human antibody is labeled anti-human IgM.

9. The method of claim 4 wherein said labeled anti-human antibody is labeled anti-human IgA.

10. The method of claim 5 wherein said beads are magnetically responsive, and steps (2) and (4) comprise exposing said first and second suspensions, respectively, to a magnetic field to draw said beads from said suspensions.

11. The method of claim 4 wherein said labeled anti-human antibody is anti-human antibody labeled with a fluorescent label.

12. The method of claim 11 wherein said fluorescent label is a member selected from the group consisting of fluorescein, fluorescein isothiocyanate, phycoerythrin, rhodamine B, and sulfonyl chloride derivative of sulforhodamine 101.

13. The method of claim 11 wherein said fluorescent label is phycoerythrin.

14. The method of claim 5 wherein said differentiation parameters are bead diameters.

15. The method of claim 5 wherein said differentiation parameters are differences in fluorescence spectra.

16. The method of claim 5 wherein said differentiation parameters are differences in light scatter.

17. The method of claim 5 wherein said differentiation parameters are differences in absorbance.

18. The method of claim 1 wherein said biological sample is a member selected from the group consisting of a blood sample, a plasma sample, and a serum sample.

19. The method of claim 1 wherein said subject tests negative for cyclic citrullinated peptide.

20. A kit for determining whether a human subject is afflicted with rheumatoid arthritis or a stage of affliction of a human subject so afflicted, said kit comprising a panel of antigens, each antigen immobilized on a solid support, said panel comprising cyclic citrullinated peptide and at least one member selected from the group consisting of
(i) BRAF1 506-525,
(ii) BRAF2 656-675,
(iii) Vimentin (protein) citrullinated,
(iv) Vimentin 415-433 cit cyclic, and
(v) Vimentin 58-77 cit3 cyclic,
(vi) Clusterin 231-250 cit sm1 cyclic,
(vii) Fibrinogen A 556-575 cit sm cyclic,
(viii) Fibrinogen A 616-635 cit sm cyclic,
(ix) Histones2A H2A/a 1-20 cit sm2 cyclic,
(x) Filaggrin 48-65 cit2v1 cyclic, and
(xi) BRAF (catalytic domain from v raf murine sarcoma viral oncogene homologue B1, amino acids 416-766),
said solid supports further bearing differentiation parameters selected such that all said supports bearing any one antigen of said panel are differentiable from all said supports bearing other antigens of said panel.

21. The kit of claim 20, wherein the panel comprises at least five members selected from the group

22. The kit of claim 20 further comprising labeled anti-human antibody.

23. The kit of claim 20 wherein said solid supports are beads.

24. The kit of claim 20 wherein said differentiation parameters are differentiable by flow cytometry.

25. The kit of claim 23 wherein said beads are magnetically responsive.

26. The kit of claim 22 wherein said labeled anti-human antibody is labeled anti-human IgG.

27. The kit of claim 22 wherein said labeled anti-human antibody is labeled anti-human IgM.

28. The kit of claim 22 wherein said labeled anti-human antibody is labeled anti-human IgA.

29. The kit of claim 22 wherein said labeled anti-human antibody is anti-human antibody labeled with a fluorescent label.

30. The kit of claim 29 wherein said fluorescent label is a member selected from the group consisting of fluorescein, fluorescein isothiocyanate, phycoerythrin, rhodamine B, and sulfonyl chloride derivative of sulforhodamine 101.

31. The kit of claim 29 wherein said fluorescent label is phycoerythrin.

32. The kit of claim 20 wherein said differentiation parameters are bead diameters.

33. The kit of claim 20 wherein said differentiation parameters differences in fluorescence spectra.

34. The kit of claim 20 wherein said differentiation parameters differences in light scatter.

35. The kit of claim 20 wherein said differentiation parameters differences in absorbance.
